(19) United States
(12) Patent Application Publication
(10) Pub. No.: US 2006/0039569 A1
(43) Pub. Date: Feb. 23, 2006

(54) Noise canceling toilet

(76) Inventor: Patrick R. Antaki, Plano, TX (US)

Correspondence Address: PATRICK ANTAKI, 1900 PRESTON ROAD #267-303, PLANO, TX, 75093, US

(21) Appl. No.: 11/207,645

(22) Filed: Aug. 19, 2005

Related U.S. Application Data
(60) Provisional application No. 60/602,967, filed on Aug. 19, 2004.

(51) Int. Cl. A61F 11/06

(52) U.S. Cl. 381/71.1

(57) ABSTRACT

A toilet acoustic noise canceling system comprising of at least one reference microphone (155) which is operatively connected to a digital signal processing circuit (240) and at least one speaker (150) which is operatively connected to same digital signal processing circuit. Digital signal processing circuit employs signal from said microphone (235) and computes in real-time an output signal (255) to said speaker (150) for the purpose of causing destructive interference of sound energy emanating from said speaker (285) with unwanted noise source acoustic energy (205) emanating in the general region of said toilet (100).

### CROSS REFERENCE TO RELATED APPLICATIONS

This application claims the benefit of provisional patent application Ser. No. 60/602,967, filed 2004 Aug. 19 by the present inventor.

### FEDERALLY SPONSORED RESEARCH

Not applicable

### SEQUENCE LISTING OR PROGRAM

Not applicable

### BACKGROUND OF THE INVENTION—FIELD OF INVENTION

This invention relates to a method of reducing acoustic energy (sound) emanating from a toilet and from its human user.

### BACKGROUND OF THE INVENTION—PRIOR ART

When a toilet is used by a human there is a loud and sometimes embarrassing noise made by the human excrements (solid, liquid or gaseous) when these are ejected from the body. Noise is also made when these excrements contact components of the toilet, such as the toilet bowl or the water surface inside the toilet. Often these sounds are amplified by resonating within the cavity enclosed on one side by the toilet bowl and on the other side by the person's exposed posterior, or even by the room in which the toilet is located. Additionally, noise is made by the toilet's flushing action, as well as by the refilling of the water tank in the toilet. Finally, noise is made by closing of the toilet seat, often when it falls onto the toilet bowl generating a large amplitude sound. All these above mentioned noises are a source of annoyance and embarrassment to persons.

Many proposals have been made to reduce various sources of noise in the toilet. Many of these proposals have focused on reducing noise emanating from flushing or from toilet use through the employment of mechanical devices to deflect human waste, muffling devices, methods of circulating water inside the toilet during flushing, and other similar contraptions. Many of these approaches are impractical to implement, particularly in the case where an already installed toilet cannot be replaced due to reasons of cost, complexity or efficacy of noise level reduction.

In the previously mentioned proposals, the method for reducing noise is passive. A passive system's primary benefit is derived from attenuating the noise energy, or in reducing the amount of noise energy generated in the first place by the physical event (such as water rushing through a valve). Due to many of these advances, modem toilets have significantly reduced the noise made by flushing and refilling of the water tank. One significant disadvantage of these techniques is that they are effective in reducing noise if the whole toilet and water supply is upgraded to this less noisy approach incorporating such techniques. This is impractical or too costly in a significant number of locations where toilets have been previously installed.

None of the general techniques mentioned above are effective in reducing human generated noises in the toilet, often the largest source of embarrassment; hence these solutions do not solve the central problem of toilet noise abatement. As an illustration of this point, it is common in Japanese toilet users to employ a special electrical button that generates a flushing sound from an electronic recording of such flushing sound (rather than from water flushing inside the toilet) in order to mask other human generated sounds. In this scenario, the toilet user wishes to emanate a loud flushing sound, thereby increasing the overall toilet noise level, rather than suppressing such sound.

An alternative approach to the problem of toilet noise reduction is to employ the concept of acoustic noise destructive interference. In this method, a sound signal of equal and opposite phase to that of the unwanted noise is generated (typically through the use of an acoustic speaker similar to ones used in audio entertainment equipment.) When combined in the air (or other physical medium), the result is a net noise level that is attenuated due to the summing of the original noise signal with the canceling signal of opposite phase and equal magnitude. This approach works well so long as the requirements of equal magnitude and opposite phase are met. However, these requirements are difficult to meet in a real-world implementation of such a system.

One inventor, Tsutsui (Japanese patent No. JP403181996A “Low flushing sound toilet stool”, Tsutsui et al), proposes to use this noise canceling method by generating a pre-recorded flushing sound (with approximately opposite phase and equal magnitude) through a speaker embedded in the toilet. The effect of generating this sound, when done at precisely the correct time, is to reduce the overall sound level due to a toilet flushing. This approach has several key problems that reduce its efficacy. First, the flushing sound which had been pre-recorded at the toilet factory varies significantly from the actual sound that emanates from the toilet flushing in an actual installation at a toilet site in use. This variation is due to the fact that the acoustics in the factory are necessarily different from a real installation (where each installation is acoustically unique), and the circumstances for flushing also vary from use to use (is the human user is sitting on the toilet or not, when flushing? The contents of the toilet bowl vary when flushing, etc.) Secondly, this approach requires a precise synchronization from the electronic payback of the recorded sound with the flushing action (which can vary significantly due to actuator aging, water pressure, etc.) Thirdly, this approach is only practical, if it was effective in the first place, with newly built toilets and is not practicable with existing installation of toilets. Finally, this approach only works for previously known sounds, namely, the flushing sound (and as has been already demonstrated, such sound tends to vary significantly over each use.) Another inventor, Suzuki (Japanese patent No. JP405132986A “Noise attenuating method for toilet bowl”, Suzuki et al), proposes to overcome some of the limitations of Tsutsui by updating the recorded flushing sound every time the toilet is flushed. Suzuki relies on a pre-recorded toilet flushing sound. This approach does enhance the fidelity of the recorded sound to that of the characteristics of each particular toilet installation, as compared to Tsutsui. However, the other problems mentioned in the Tsutsui patent remain. Suzuki mentions in his patent's Claim #1 Paragraph #5 that it is difficult to attenuate the flushing sound after the flushing sound has started, thereby prescribing the use of the pre-recorded flushing sound cancellation method.

Both Suzuki and Tsutsui affirm the utility of applying active noise cancellation techniques to attenuate noise emanating from toilets. However, they both fall short of an effective and useful method for doing so.

### BACKGROUND OF THE INVENTION—OBJECTS AND ADVANTAGES

The present invention described below solves the problem to which Suzuki alludes, and thus applies the solution not only to flushing sounds, but to all other sounds produced inside the toilet.

Accordingly, several objects and advantages of this invention are that toilet noise is reduced, regardless of its source. The invention reduces noise energy, and therefore the perceived loudness of that noise, emanating from the flushing action, from the water tank filling, from human generated noises (solid, liquid, and gaseous), and from ancillary toilet noise sources (such as the falling of the toilet seat onto the toilet bowl). The previously mentioned techniques are limited in their effectiveness to specific noise sources, are thus are not effective, or are simply inoperable, against the broader spectrum of noise sources emanating from within the toilet and its user. Additionally, the character of the noise sources to be canceled by the invention includes both transient and steady-state elements. It is generally easier to attenuate steady-state noise sources, as they are, by definition, repetitive (and thus one can use historical parameters of the steady-state components of the noise to generate a cancellation signal at the present time). As for canceling the transient components of the noise, a real-time adaptive approach is required for efficacy. The invention describes this approach.

Other objects and advantages of this invention are that the apparatus embodying the invention may be installed and utilized onto existing toilets, without the need to replace the toilet or to modify any plumbing. The apparatus embodying the invention is relatively small as compared to the typical dimensions of the western style toilet bowl, and thus is not difficult to retrofit onto existing toilets. The previously mentioned techniques typically require a significant (and costly) modification to the toilet, including often the complete replacement of the toilet and related plumbing accessories.

Another object and advantage of the invention is to reduce the noise energy emanating from the toilet regardless of the noise's characteristics, which may vary with time, with temperature, with the level of humidity, with the level of water pressure, with the amount and type of waste in the toilet, with the acoustic characteristics of the rest of the room where the toilet is located, and with the physical characteristics and position (relative to the toilet) of the human currently using the toilet, and other acoustic variations. The previously mentioned techniques related to electronic noise cancellation are ineffective against human made noises, and do not accommodate changing acoustic properties of the toilet, its user and the surroundings.

### SUMMARY

The invention consists of the application of adaptive and active electronic noise cancellation techniques in the toilet bowl. One or more acoustic speakers (electrical to acoustical energy transducers) are located within the toilet bowl, in close proximity to the majority of noise sources to be attenuated. The speaker generates acoustic waves that interfere destructively with the noise to be attenuated. The net sound energy, resulting from the interference of the speaker with the noise source, is attenuated as compared to the original noise source. The speaker is electrically stimulated from an electronic circuit containing a digital signal processing circuit, one or more digital-to-analog converters and one or more analog-to-digital converters. The digital signal processor processes the sound energy received from one or more microphones (acoustic to electrical energy transducers) in order to compute the appropriate output signal to be sent to the speaker via the digital-to-analog converter(s) and amplifier(s). At least one microphone is located in close proximity to the noise source to be attenuated.

### DRAWINGS—FIGURES

FIG. 1 shows the preferred embodiment of the invention attached to the toilet

FIG. 2 shows the functional block diagram of the invention

FIG. 3 shows a block diagram of the digital signal processing circuit computation

### DRAWINGS—REFERENCE NUMERALS

100—Toilet bowl
105—Inside surface of toilet bowl
110—Toilet seat
115—Back portion of toilet
120—Toilet seat hinge
125—Toilet seat flange
130—Toilet seat bolt
135—Toilet seat nut
140—Invention attachment bracket
145—Invention enclosure
150—Primary acoustic speaker
155—Acoustic reference microphone
160—Invention printed circuit board
165—Invention electrical cable
170—Invention AC-to-DC power supply
175—AC power plug
180—Acoustic error microphone
200—Noise source to be attenuated
205—Propagating acoustic energy from noise source
210—Impinging acoustic energy into reference microphone
215—Impinging acoustic energy into error microphone
217—Analog electrical signal from reference microphone
218—Analog electrical signal from error microphone
220—Low pass filter electronic circuit
225—Filtered analog reference microphone electrical signal
230—Analog to digital converter circuit
235—Digital reference microphone electrical signal
236—Digital error microphone electrical signal
240—Digital signal processing circuit
255—Digital signal of primary acoustic speaker
256—Digital signal of second acoustic speaker
260—Digital to analog converter circuit
265—Analog signal of primary acoustic speaker
270—Amplifier circuit
275—Amplified analog signal of primary acoustic speaker
280—Second acoustic speaker
285—Acoustic energy emanating from primary speaker
290—Acoustic energy emanating from second speaker
295—Electrical power source
300—Reference path filter
305—Adaptation calculator
310—Error path filter
320—Summer

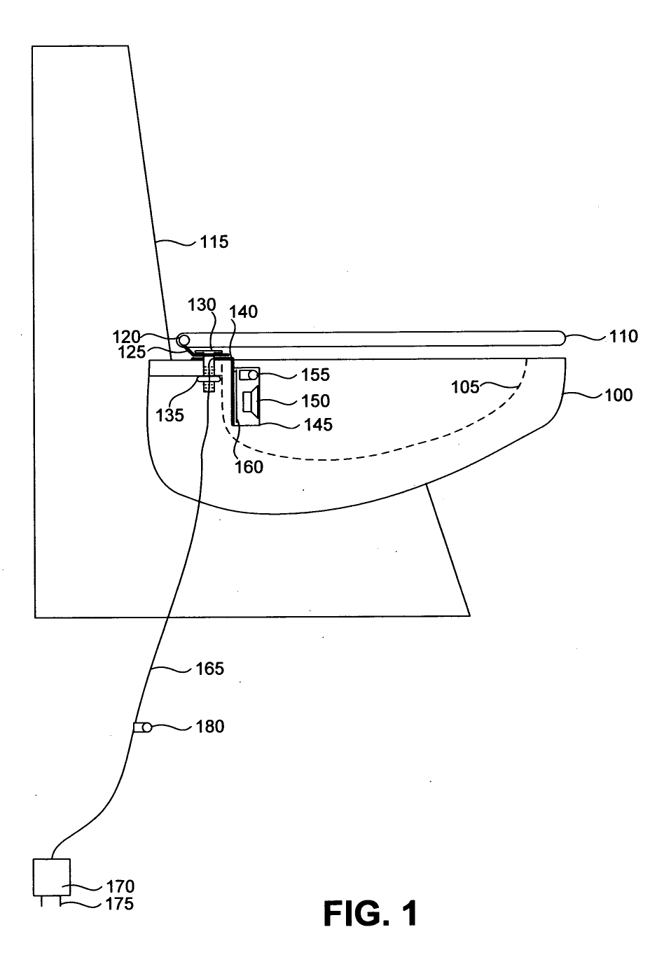

### DETAILED DESCRIPTION—FIGS. 1-3—PREFERRED EMBODIMENT

A preferred embodiment of the physical placement of the major components of the invention is illustrated in FIG. 1. The invention enclosure 145 is attached to toilet bowl 100 through bracket 140. Electrical power is provided to invention printed circuit board 160 through electrical cable 165. Cable 165 is routed through hollowed out toilet seat bolt 130. This arrangement enables an electrical connection to be routed from inside toilet bowl 100 to outside toilet bowl region. Cable 165 also contains electrical wires for error microphone 180 which is located outside toilet 100. Toilet seat 110 is attached to toilet bowl 100 through toilet seat hinge 120 and toilet seat flange 125 enabling seat 110 to rotate up to back portion of toilet 115 and down to toilet 100. Toilet seat hinge 120 and toilet seat flange 125 are typical components of toilet seat 110, and are made to enable the replacement of toilet seat 110 from toilet 100. Toilet seat bolt 130 is secured to toilet 100 with toilet seat nut 135. This overall attachment arrangement allows for the rigid placement of invention enclosure 145 inside toilet 100, where embedded acoustic reference microphone 155, primary acoustic speaker 150 and invention printed circuit board 160 are most effective for their task to reduce the total noise energy emanated from toilet 100. Invention AC-to-DC power supply 170 connects to the main AC power source in the vicinity of toilet 100 through AC power plug 175, thus providing electrical power to the rest of the invention. The location of acoustic error microphone 180 outside toilet 100 is important in that it is located relatively far from the main noise source, thus enabling effective adaptation of the noise cancellation computation to changing environmental conditions. Likewise, the location of acoustic reference microphone 155 and primary acoustic speaker 150 very close to the primary noise source inside toilet 110 and close to inner surface of toilet bowl 105 enables the effective attenuation of noise sources emanating from this region.

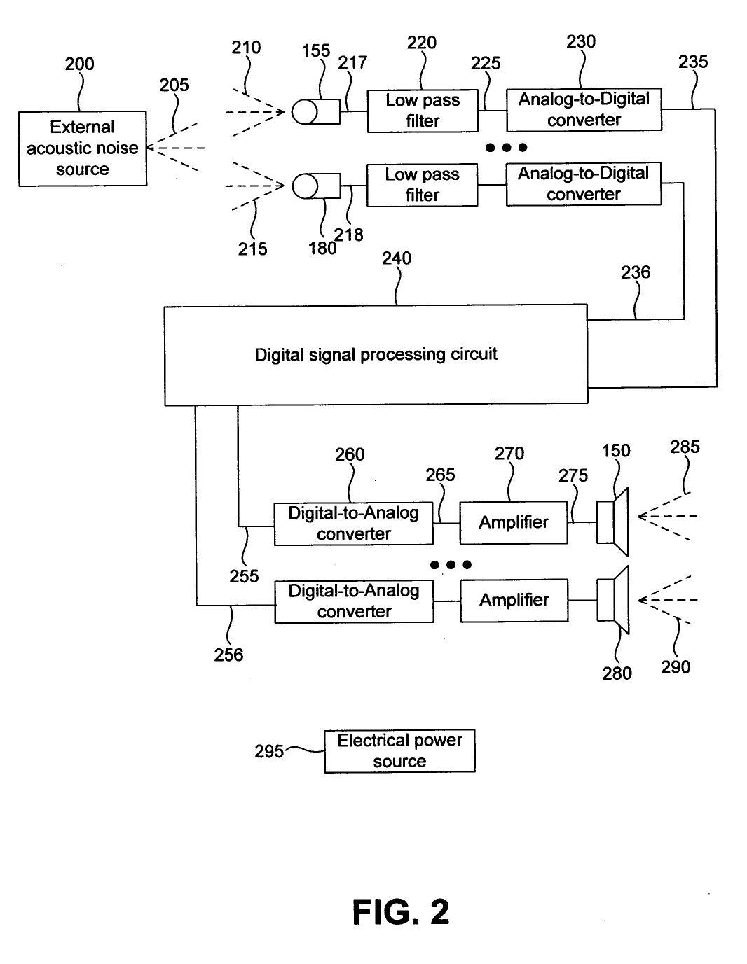

A preferred embodiment of the present invention's logical block diagram of the major components is illustrated in FIG. 2. External acoustic noise source 200 generates propagating acoustic energy 205. The object of the invention is to minimize the effective energy of the propagating acoustic energy from noise source 205 that impinges outside toilet 100. Acoustic reference microphone 155 converts impinging acoustic energy into reference microphone 210 and converts that energy into analog electrical signal 217. Likewise, acoustic error microphone 215 converts impinging acoustic energy into error microphone 180 and converts that energy into analog electrical signal 218. Analog signal 217 is passed through low-pass filter electronic circuit 220 (also known as an anti-aliasing filter). The resulting filtered analog signal 225 is converted into digital signal 235 by analog to digital converter circuit 230. In a similar arrangement, analog signal from error microphone 218 goes through a similar set of circuits until resulting digital signal from error microphone 236 is available for use by digital signal processing circuit 240. Digital signal processing circuit 240 receives digital signals from reference microphone 235 and error microphone 236. This circuit performs various computations upon its input signals and generates digital output electrical signal 255. This digital signal is converted to analog signal of primary acoustic speaker 265 through analog to digital converter 260. Amplifier 270 electrically amplifies analog signal 265 and creates amplified analog signal 275. Amplified analog signal of primary acoustic speaker 275 is fed into primary acoustic speaker 150 to create acoustic energy emanating from primary speaker 285. Electrical power source 295 provides electrical power for the operation of the invention.

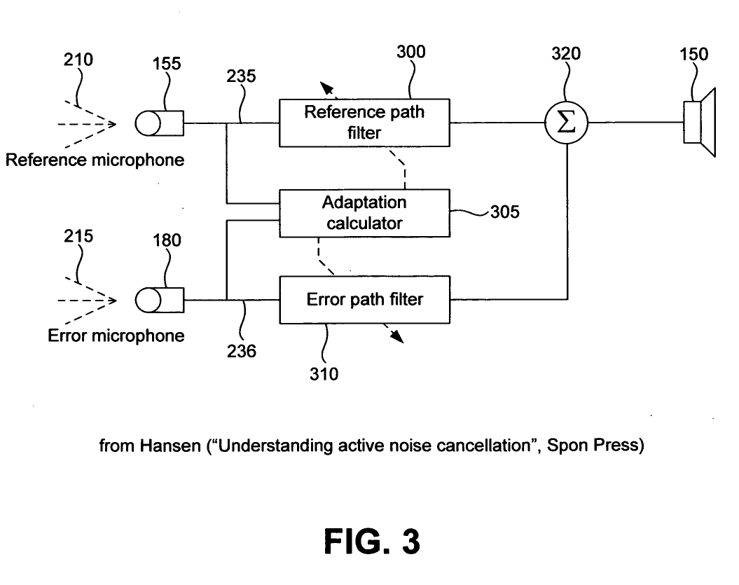

A preferred embodiment of the present invention's digital signal processing circuit computation is shown in FIG. 3. Digital reference microphone electrical signal 235 (Reference Signal) contains a representation of impinging acoustic energy into reference microphone 210. Digital error microphone electrical signal 236 contains a representation of impinging acoustic energy into error microphone 215 (Error Signal). Reference path filter 300 and error path filter 310 create signals that are algebraically summed in summer 320 prior to being provided to primary acoustic speaker 150. Reference path filter 300 computes a filter function using Reference Signal 235 as its input. Error path filter 310 computes a filter function using Error Signal 236 as its input. Adaptation calculator 305 utilizes both Reference Signal 235 and Error Signal 236 in order to compute new filter characteristics of reference path filter 300 and of error path filter 310. Note that reference path filter 300 and error path filter 310 are each a unique filter, as each of these performs a different function in the overall noise cancellation computation. Changes to this filter characteristics occur frequently during the noise cancellation process, on the order of approximately 10 to 100 times per second. The use of adaptation calculator 305 to effect changes in reference path filter 300 and error path filter 310 is one critical element in the efficacy of the invention in maximizing the attenuation of noise acoustic energy 205. Acoustic energy emanating from primary speaker 285 is combined with Propagating acoustic energy from noise source 205 in the air. The resulting combination appears at the input of reference microphone 155 as impinging acoustic energy into reference microphone 210. Likewise, a different combination (due to the physically different location of error microphone 180) of acoustic energy emanating from primary speaker 285 and acoustic energy from noise source 205 appears at the input of error microphone 180 as impinging acoustic energy into error microphone 215. These inputs to both microphones form a closed loop feedback system through the acoustic path.

### ALTERNATIVE EMBODIMENTS

In an alternative embodiment of the invention, only a single acoustic microphone, namely acoustic reference microphone 155, may be utilized. In this embodiment, the resulting performance of the invention, as measured by the amount of audible noise cancellation of the noise source is somewhat reduced as compared to the use of at least one additional microphone, namely acoustic error microphone 180. Correspondingly, the computations performed by digital signal processing circuit 240 in a single-microphone embodiment is less complex than in two or more microphone embodiments.

In an alternative embodiment of the invention, a multiplicity of acoustic speakers may be utilized to improve the noise cancellation performance of the invention. FIG. 1 shows the addition of second acoustic speaker 280 generating acoustic energy 290. Second speaker digital signal 256 is generated from the computations inside digital signal processing circuit 240 in a similar manner as digital signal of primary acoustic speaker 255. Note that the digital signals and resulting acoustic energy emanating from the multiplicity of speakers need not be identical. It is possible that the physical location and installation of the speakers be made such that one is inside toilet 100, and one is outside, or other combinations thereof. Such arrangements would necessitate that the computations in digital signal processing circuit 240 be different for each output speaker in order to optimize the efficacy of the invention's operation.

In an alternative embodiment of the invention, electrical power may be provided to the invention from sources other than AC power (typical wall outlet). For example, a battery may be used. This battery may be of the primary type whereby it is not rechargeable and must be replaced when it has been drained of its capacity. Alternatively, such a battery may be of the rechargeable type. A multiplicity of battery recharging methods may be utilized to provide power to the invention. Some of these methods include the use of light-to-electrical converters (similar to solar cells), AC power (in this case the battery continues to provide electrical power to the invention even if AC power is temporarily unavailable), mechanical-to-electrical power derived from the movement of water during the flushing of the toilet or the refilling of the toilet's tank.

In an alternative embodiment of the invention, invention enclosure 145 containing invention printed circuit board 160 may be located physically outside toilet 100, or in another location within toilet 100. The location of reference microphone 155 and primary acoustic speaker 150 must be located within the confines of toilet 100, but may be moved from the location shown in the preferred embodiment of the invention to another location. An alternative location may be on the inner side of toilet 100, embedded at the time of manufacturing toilet 100 within the toilet structure (such as in a cavity in the plastic or ceramic make-up of toilet 100), or inside back portion of toilet 115. In the case where toilet 100 is designed and manufactured to accommodate the invention internally, it is possible to use electrical power cables and electrical power sources that may be used in toilet 100 for other functions unrelated to the invention (such as automatic flushers, toilet seat or water warmers, and a variety of other toilet functions). The location of error microphone 180 may be separated from invention electrical cable 165 and moved to its own separate location, either physically separated from toilet 100 with a separate cable (or wireless connection), or embedded within toilet 100 or back portion of toilet 115 in a manner that is as far away physically as practical from acoustic noise source 200. It is important that any components of the invention that are placed within toilet 100 be protected in an enclosure that is waterproof as well as resilient to cleaning from brushes or such implements as might be used to clean toilet 100.

How Acoustic Noise Cancellation Works

Acoustic noise cancellation works by the interference of the acoustic noise energy with the speaker acoustic energy. If the speaker generates the same acoustic signal as the noise, but with opposite phase and equal magnitude, then these two signals interfere destructively, resulting in a net energy that is close to zero (hence, silence). Some of the key factors contributing to a less-than-perfect acoustic noise cancellation are the distance of the speaker from the noise source, the time delay that the electronic system requires in order to generate an opposite phase acoustic signal, and the fidelity of the electro-acoustic components (microphones, speakers, resonant chamber made up of the toilet system) including their time-changing properties. In a real-world implementation of an acoustic noise canceling system, all three of these factors interact and affect how the designer might trade-off their respective implementation.

Below, each of these three major critical performance parameters is examined as well as how the invention copes with these issues.

Distance of the speaker to the noise source. The wavelength of sound at 600 Hz is approximately 1.9 feet (at room temperature and sea level conditions). In order for the noise cancellation to be effective (i.e., greater than a 10 dB reduction in noise level), it is important that the distance between the two sources of noise be less than 0.2 wavelengths (Hansen “Understanding active noise cancellation”, Spon Press). Therefore, in order for the 600 Hz noise component to be sufficiently attenuated for a human to perceive such attenuation, a maximum distance of 4.5 inches is required. The bulk of the noise energy of interest to the invention lies in the lower frequency spectrum of human hearing; hence a 600 Hz upper limit is reasonable for purposes of calculating desired maximum distances. The invention places the primary acoustic speaker within such a distance inside the toilet. A related concern is the location of the reference acoustic microphone. The location of this microphone is also critical in efficacy of the noise canceling system performance. The invention also locates this microphone close to the noise source. If an error microphone is used, then it is equally critical that the error microphone be located relatively far from the noise source, namely, at least one wavelength away. The invention places the error microphone far from the inside of toilet bowl by attaching it at some distance from the invention enclosure on the invention electrical cable. Finally, it is critical to note that the acoustic properties of the inside of the toilet bowl change with each use, as the physical properties (size, shape, temperature, humidity, and a multiplicity of such factors) of the acoustic chamber also change. Therefore the location of the acoustic elements (reference microphone and primary speaker) within the toilet ensures that the proper electro-acoustic conditions are in place for the invention's efficacy. Placing these elements outside the toilet leads to significantly lesser noise cancellation, particularly at the higher frequencies.

Time delay to generate the opposite phase acoustic signal. The active noise cancellation approach of the invention uses an adaptive technique. Unlike the Suzuki and Tsutsui patents, the invention does not store a previously recorded digital replica of the noise to be cancelled (in the other patents' cases, they store the flushing sound only, ignoring the other human-generated sounds). The invention relies on the real-time computation of the acoustic signals detected by the reference and error microphones in order to generate the canceling signal of the primary speaker. Therefore, the total time delay from when the noise source originally produces a particular sound energy, until the primary speaker's acoustic corresponding energy interferes in the physical medium (most of the interference of the two energy sources occurs within the air inside the toilet bowl) is a very critical parameter in determining the efficacy of the invention. The time delay is measured against the period of the frequency of the noise spectral component to be cancelled. Hence, a particular time delay results in worse performance (due to a greater phase shift) at the higher frequencies than at the lower frequencies. The first major contributing time delay is that of the sound propagation from the noise source to the reference microphone, and subsequently from the primary speaker to the noise source where the in-air interference can ideally take place. The speed of sound is approximately 331 meters/sec. Assuming that the distance from the noise source to the primary microphone is 4.5 inches, the sound takes approximately 0.34 milliseconds to travel such distance. This time delay is equivalent to 0.2 times the period of a 600 Hz noise component (or almost 75 degrees of phase shift). By taking into account the time delay of the sound emanating from the primary speaker (which is almost identical to that of the noise-to-microphone delay), it is reasonable then to double the overall delay to 0.68 milliseconds. Other sources of delay are the phase shifts inherent in the construction of the microphone and of the speaker. The preferred embodiment of the invention uses a MEMS microphone construction and an Ultrasonic-response speaker (both of which exhibit a fast response time and thus lower delay). The reason for these preferred choices are to minimize the time delays contributed by these two elements. The internal circuitry of the invention also contributes some delays to the overall system. The contributions to delay of the low pass filters, analog to digital converters, digital to analog converters, and amplifiers are generally negligible. The last contributor to system delay is the computation performed within the digital signal processing circuit. That delay is a function of the complexity of the computation, the speed of the digital signal processing circuit, as well as the inherent algorithmic delays inherent in the computation (for example, the inherent phase shift due to a digital filter implementation in the digital signal processing circuit, such phase shift being due to the nature of the filter characteristics rather than the complexity of its computation). The invention mitigates the signal processing delay by using a modern digital signal processing circuit capable of high speed operation (on the order of 100 MHz basic digital clock rate).

Fidelity of acoustic components and their time-changing properties. The acoustic characteristics of the main elements that make-up the noise canceling system have a significant impact on the efficacy of the invention. The major factors affecting the acoustic characteristics of the system include:

a) initial manufacturing part-to-part tolerance of the electro-acoustic components (microphones, speakers)

b) changes of electro-acoustic components' parameters due to aging

c) installation (location and orientation) of invention within the toilet as well as the location of the error microphone

d) toilet bowl and toilet seat properties (size, shape material, construction, attachment)

e) surrounding region of the toilet (size of room sound dampening or resonant characteristics)

f) external changes to the acoustic coupling (for example, water or other foreign materials on the surface of the invention enclosure)

g) temperature and humidity

h) changes to the acoustic characteristics of the toilet system that occur with each use of the invention. This includes the water level inside the toilet, foreign materials inside the toilet (level, shape, type), the human using the toilet (location, size, shape, orientation, clothing), the toilet seat (location and orientation), and the changes to the surrounding region of the toilet (such as open or closed windows, changes in the contents of the room, curtains open or closed), character of acoustic energy sources outside the toilet and their coupling to the inside of the toilet.

The invention copes with the large number and unpredictability of possible variations in the acoustic environment with the use of (a) an adaptive computation of the digital signal of primary acoustic speaker, (b) an error microphone, and (c) a closed-loop computation as shown in FIG. 3. The invention does not make use of a pre-stored recording of noise sources, even if such recordings are continuously modified and adapted, since the use-to-use changes are unpredictable, and these do not take into account the human contributions to the noise.

### ADVANTAGES

From the description above, a number of advantages of the present invention become evident:

a) The volume of undesired noise generated within the region of the toilet is actively reduced, including both flushing noise as well as noise emanating from a human user of the toilet.

b) The invention may be added to existing toilets, without the need for significant replacements of toilets, plumbing, or other structural changes. Additionally, the invention may also be embedded into the design and manufacture of a new model toilet for new installations, rather than a retrofit of a toilet.

c) The invention works all the time, it does not require a triggering event (such as an input from the flushing system) to activate it.

d) The invention is small enough to be located in an effective region with respect to proximity to noise sources, but does not interfere with the normal operation of a toilet.

e) The invention adapts automatically and often to changes in the acoustic environment of the toilet and its surroundings and users, hence maximizing its efficacy in a greater spectrum of operating and installation situations.

### DEFINITIONS

Toilet. This term refers to any apparatus intended to collect the excrements during defecation or urination by humans.
Inside toilet. This term refers to the general region of a toilet where the human excrements are initially deposited during defecation or urination.
General region of toilet. This term refers to the vicinity around the toilet, such as the space in the room where the toilet bowl is located. From an acoustic standpoint, this refers to the region where the noise level is not perceptively attenuated (when noise cancellation is not in use), as perceived by a human, as compared to the noise level at its point of origin.
Noise. This term refers to human audible sounds made immediately before, during, and immediately after the defecation or urination. The object of the invention is to attenuate the audible level of such noise, since this noise represents unwanted sound.
Noise canceling. This is action of the invention system by causing destructive interference with the noise. The degree to which noise canceling occurs varies, and may not be absolute (i.e., total silence).
Microphone. This term refers to an acoustic energy to electrical energy transducer.
Speaker. This term refers to an electrical energy to acoustic energy transducer.
Microphone operatively connected. This term refers to the intent of the connection. In the case of a microphone connection to a digital signal processing circuit, the operative connection includes any circuit, including electrical wires, that converts the microphone's electrical signal output (resulting from an impinging acoustic energy onto the microphone's active surface) into a usable digital format.
Speaker operatively connected. This term refers to the intent of the connection. In the case of a speaker connection to a digital signal processing circuit, the operative connection includes any circuit, including electrical wires, that converts the digital signal processing circuit's digital data output intended for the speaker into a usable electrical signal directly fed into the speaker's electrical inputs, generating an acoustic energy output from the speaker's active surface.
Digital signal processing circuit. This term refers to a digital circuit that performs a series of mathematical operations on its inputs (in this case, the microphone signals) and creates output signals (in this case, the speaker signals). A multiplicity of methods for implementing a digital signal processing circuit is possible.
In real-time. This term refers to the time elapsed to perform certain computations. With respect to the invention, computing in real-time refers to the concept that the time for performing such computations is shorter in duration than the time of the overall transaction (that is, the duration of each toilet noise cancellation usage event, which is the approximate duration of the time for a human to use the toilet for each instance of defecation, urination, flushing the toilet, or of refilling the water tank). An example of non real-time computation is given by the Suzuki patent, whereby the computation (in this case, the recording) of the canceling speaker signal is based upon the last time the user flushed the toilet, rather than on the current use of the toilet.
Destructive interference. This term refers to the natural action of sound energy in air or other sound-transporting media (such as the ceramic material of a toilet). Destructive interference occurs when two or more sound energy signals from distinct sources combine in a manner so as to reduce the overall resulting energy after such combination. Such reduction in total energy occurs if the phase of each signal is opposite that of the other signal's phase, and the magnitudes of each signal are equal. In order for the destructive interference to be very effective, the phase ad magnitude criteria mention previously must be true for all frequencies during the complete duration of the noise cancellation event, as well as for all spatial locations in the toilet.
Characteristics of the computation. The digital signal processing circuit performs certain computations including (but not limited to) one or more digital filters (conventionally known in engineering as FIR, IIR or other similar filter configurations). The characteristics of the computation refer primarily to the filter type, filter architecture, filter coefficients, number of taps, and similar related parameters employed in the computation performed by the digital signal processor circuit in the invention.
Wavelength of sound. This term refers to the physical distance that is represented by one wavelength of sound at a given frequency, under typically normal operating conditions for the invention. For example, the wavelength of sound at 20C, sea level, for 600 Hz is approximately 23 inches. Noise typically contains a multitude of sound frequencies, each exhibiting a distinct wavelength. The highest frequency exhibits the shortest wavelength. Therefore, the highest frequency of interest (for purposes of canceling the noise) is the determining factor in the maximum operable distance in the design of a noise canceling system.
Spatially close. In reference to the present invention, this term signifies that the distance between the referring items is equal to or less than one wavelength of sound.
Spatially far. In reference to the present invention, this term signifies that the distance between the referring items is greater than one wavelength of sound.

What is claimed is:

1. A toilet noise canceling system comprising: a microphone which is operatively connected to a digital signal processing circuit, a speaker which is operatively connected to same digital signal processing circuit, said digital signal processing circuit employing signal from said microphone and computing in real-time operative signal to said speaker for the purpose of causing destructive interference of sound emanating from said speaker with unwanted noise source or sources emanating in the general region of said toilet.

2. The system of claim 1, wherein said microphone and speaker are located inside said toilet.

3. The system of claim 1, wherein said microphone is located within a distance of less than the length of one wavelength of sound for the highest frequency of sound desired to be attenuated by said system.

4. The system of claim 1, wherein said speaker is located within a distance of less than the length of one wavelength of sound for the highest frequency of sound desired to be attenuated by said system.

5. The system of claim 1, wherein a multiplicity of microphones are operatively connected to same digital signal processing circuit.

6. The system of claim 1, wherein a multiplicity of speakers are operatively connected to same digital signal processing circuit.

7. The system of claim 1, wherein the computation of the digital signal processing circuit modifies characteristics of the computation periodically.

8. The system of claim 1, wherein the computation of the digital signal processing circuit modifies characteristics of the computation more frequently than once per second.

9. The system of claim 1, wherein a multiplicity of microphones are operatively connected to same digital signal processing circuit, and the computation of the digital signal processing circuit modifies characteristics of the computation.

10. The system of claim 1, wherein a multiplicity of microphones are operatively connected to same digital signal processing circuit, and the computation of the digital signal processing circuit modifies characteristics of the computation, and the amount of influence of each said microphone onto said modification is uniquely computed.

11. A method of attenuating unwanted acoustic noise emanating from the general region of a toilet, comprising of the steps of:
a) providing for a means to detect unwanted acoustic energy in the region of a toilet,
b) a means of converting said unwanted acoustic energy into a representative digital signal,
c) a means of performing computations using said digital signal and calculating one or more resulting output signals in real-time,
d) a means of converting the computed resulting output signal or signals into interfering acoustic energy,
e) a means of causing interference between the unwanted acoustic energy and the interfering acoustic energy in a manner that is spatially close to source of said unwanted acoustic energy,
f) a means of detecting the resulting acoustic energy resulting from said interference,
g) a means of converting said resulting acoustic energy into a representative digital error signal,
h) a means of performing computations using said digital error signal to modify said output signal or signals in real-time,
whereby the audible level of unwanted acoustic energy emanating from the general region of a toilet is reduced.

12. The method of claim 11, wherein said means to detect unwanted acoustic energy is located spatially close to said source of noise.

13. The method of claim 11, wherein a multiplicity of microphones are used to detect unwanted acoustic energy.

14. The method of claim 11, wherein a multiplicity of microphones are used to detect unwanted acoustic energy, and at least one microphone is located spatially close to said source of unwanted acoustic energy.

15. The method of claim 11, wherein a multiplicity of microphones are used to detect unwanted acoustic energy, and at least one microphone is located spatially close to said source of unwanted acoustic energy, and at least one microphone is located spatially far from said source of unwanted acoustic energy.

16. The method of claim 11, wherein said means to convey resulting output signal or signals into interfering acoustic energy is performed through the use of one or more speakers.

17. The method of claim 11, wherein said means to perform computations to generate the output signal or signals is changed periodically based upon information conveyed from the means to detect unwanted acoustic energy.

18. The method of claim 11, wherein a multiplicity of microphones are used to detect unwanted acoustic energy, and said means to perform computations to generate the output signal or signals is changed periodically based upon information conveyed from each microphone.

19. The method of claim 11, wherein a multiplicity of microphones are used to detect unwanted acoustic energy, and said means to perform computations to generate the output signal or signals is changed periodically based upon information conveyed from each microphone, and the amount of influence of each said microphone onto said modification is uniquely computed.
